(19) United States
(12) Patent Application Publication
(10) Pub. No.: US 2021/0298674 A1
(43) Pub. Date: Sep. 30, 2021

(54) METHOD FOR REGISTRATION AND ACTIVATION OF TEMPERATURE-SENSING GARMENTS

(71) Applicant: Siren Care, Inc., San Francisco, CA (US)

(72) Inventors: Jie FU, Shanghai (CN); Xuening SHEN, Shanghai (CN)

(21) Appl. No.: 17/346,176

(22) Filed: Jun. 11, 2021

Related U.S. Application Data
(63) Continuation of application No. PCT/CN2018/121246, filed on Dec. 14, 2018.

(51) Int. Cl. A61B 5/00; A61B 5/01; A41B 11/00

(52) U.S. Cl. CPC A61B 5/6807; A61B 2562/04; A41B 11/00; A61B 5/01

(57) ABSTRACT

A system for monitoring a user may include a garment configured to be placed on a foot of the user, a packaging sensor coupled to the garment and configured to detect separation of the garment and packaging associated with the garment, and at least one processor configured to transition the garment from an inactive state to an active state at least partially based on the separation of the garment and packaging. A method for operating a temperature-sensing garment includes operating the garment in an inactive state, detecting separation of the garment and packaging associated with the garment, predicting that the garment is placed on a user, and transitioning the garment from the inactive state to an active state in which the garment is configured to measure at least one temperature of the user.

### TECHNICAL FIELD

This invention relates generally to the field of foot care and more specifically to new and useful temperature-sensing garments and a method in the field of foot care.

### BACKGROUND

Diabetes is an increasingly common medical condition in which the body has an impaired ability to produce or respond to the hormone insulin. Diabetes damages blood vessels and nerves, particularly in the feet, and can lead to severe medical complications that are difficult to treat. For example, one complication of poorly controlled diabetes is foot ulcers, which may fail to heal because of poor blood circulation in diabetics and because treatment does not always successfully halt the spread of infection. Diabetic foot ulcers are painful and, when unresolved, can lead to lower limb amputations. As another example, Charcot foot, also known as Charcot arthropathy, is a debilitating complication of diabetes involving fractures and dislocations of bones and joints that occur with minimal or no known trauma.

Self-care is critical to detecting early signs of ulcers, Charcot foot, and other injuries and allowing timely treatment. However, visual inspection for detecting such conditions has limitations. For example, obese or visually impaired patients may not be able to see their feet easily. Additionally, due to neuropathy (numbness or loss of feeling as a result of nerve damage) caused by diabetes, a patient may not be able to feel early development of a foot ulcer and/or fractures. Even further, X-rays are unable to reliably show early stages of fractures. Accordingly, painful and dangerous foot conditions may be detected only when they have progressed to a more severe state, which increases the likelihood of extreme treatments such as amputation.

Thus, there is a need for new and improved systems and methods for monitoring the feet health of patients.

### SUMMARY

Generally, a system for monitoring a user may include a garment configured to be placed on a foot of the user, a packaging sensor coupled to the garment and configured to detect separation of the garment and packaging associated with the garment, and at least one processor configured to transition the garment from an inactive state to an active state, where the transition is at least partially based on the detection of separation of the garment and packaging. In some variations, the garment may include at least one temperature sensor to measure at least one temperature on the foot of the user. For example, the garment may include a sock and a plurality of temperature sensors to measure temperatures on multiple locations of the foot of the user. The garment may also include at least one power source configured to provide electrical power to the temperature sensor(s) and other electrical components.

When the garment is in the inactive state, the at least one processor may be configured to reduce use of energy from the power source. The garment may be maintained in the inactive state, for example, when the garment is in storage and/or in transport prior to being worn by a user. Accordingly, the inactive state may help prolong battery life and associated shelf life of the garment. When the garment is in active state, the at least one processor may be configured to receive temperature data from one or more temperature sensors.

The transition between the inactive state and the active state of the garment may be at least partially based on detection of separation of the garment and packaging. In some variations, the packaging sensor may include a Hall effect sensor and the packaging may include at least one magnet configured to generate a magnetic field proximate the Hall effect sensor. Presence or absence of the magnetic field (detectable by the packaging sensor) may be correlated with presence or absence of the packaging. Additionally or alternatively, in some variations the packaging sensor may include a light sensor configured to detect light when the garment is separated from the packaging (e.g., when the light sensor becomes exposed to detectable light). In some variations, the packaging can include a sleeve or similar component configured to wrap at least partially around the garment.

In some variations, the garment may further be paired with an electronic device such that the garment communicates temperature data to the electronic device when the garment is in the active state. For example, the electronic device may be a hub, mobile computing device, or other suitable computing device. The pairing may occur before shipment of the garment, and/or may occur after the garment transitions to its active state.

Generally, a method for operating a temperature-sensing garment includes operating the garment in an inactive state, detecting separation of the garment and packaging associated with the garment, predicting that the garment is placed on a user, and transitioning the garment from the inactive state to an active state in which the garment is configured to measure at least one temperature of the user. In some variations, the method may further include pairing the garment with an electronic device such as a hub, mobile computing device, or other suitable computing device.

In some variations, detecting separation of the garment and packaging comprises detecting removal of a magnetic field. Additionally or alternatively, detecting separation of the garment and packaging comprises detecting light when the garment is separated from the packaging. Furthermore, in some variations, predicting that the garment is placed on a user includes detecting motion and/or a predetermined orientation of the garment. Detection of the separation between the garment and packaging, and/or prediction that the garment is placed on the user, may be used as factors to transition the garment to the active state.

The inactive state may be a low-energy use state compared to the active state, in which temperature data may be communicated to the electronic device. In some variations, the garment may be operated in the inactive state during storage and/or transport of the garment, and the garment may be operated in the active state when the garment is determined to be worn by the user.

### BRIEF DESCRIPTION OF THE DRAWINGS

FIG. 1 is a flowchart representation of a method for registration and activation of a temperature-sensing garment.

FIG. 2 is a flowchart representation of a variation of a method for registration and activation of a temperature-sensing garment.

FIG. 3 is a flowchart representation of a variation of a method for monitoring feet health of a user.

FIG. 4 is a flowchart representation of a variation of a method for monitoring feet health of a user.

FIG. 5 is a flowchart representation of a variation of a method for monitoring feet health of a user.

FIG. 6 is a flowchart representation of a variation of a method for monitoring feet health of a user.

FIG. 7 is a schematic illustration of an exemplary variation of a temperature-sensing garment.

FIG. 8 is a schematic illustration of an exemplary variation of a portion of a temperature-sensing garment.

FIGS. 9A and 9B are schematic illustrations of an exemplary variation of an arrangement for detecting separation of a garment and packaging, such as for use in activating the garment.

### DETAILED DESCRIPTION

Non-limiting examples of various aspects and variations of the invention are described herein and illustrated in the accompanying drawings.

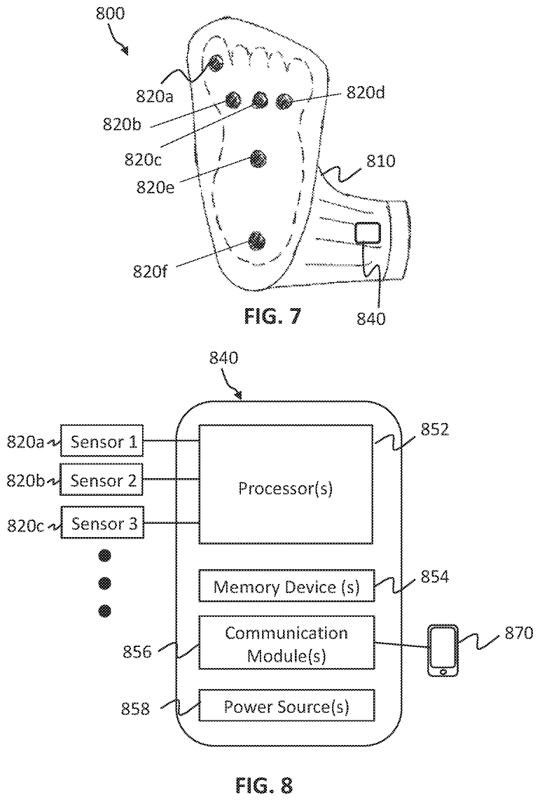

Described herein are systems and methods for registering and/or activating temperature-sensing garments. As shown generally in FIG. 7, in some variations, a user monitoring system 800 may include at least one garment 810 configured to be placed or worn on a foot of the user. The garment is also referred to herein as a sock. However, the garment may alternatively be any suitable component to be positioned on the foot such as a shoe, a slipper, an insole, etc. The garment may be configured specifically for a left foot (e.g., include a toe box accommodating contours of a left foot), for a right foot (e.g., include a toe box accommodating contours of a right foot), or may be universally or suitable for both feet. The garment may include one or more labels that are sewn, woven, or otherwise incorporated into or coupled to the garment. Examples of labels include an indication of left or right foot compatibility, size (e.g., small, medium, large, or numeric size), or other identifying info.

The garment 810 may be configured to be worn on a foot (e.g., of a human) and include a set of one or more sensors integrated into or otherwise attached to the garment. The sensors may be configured to measure one or more physical characteristics of the user, such as skin temperatures at one or more locations on a foot of the user. In some variations, a user may wear two garments, including one garment on a left foot of the user, and another garment on a right foot of the user. Temperature measurements may be performed substantially continuously as the garments are worn. In some variations, the temperature-sensing garment can be paired (i.e., communicatively coupled) with a matching temperature-sensing garment (e.g., “a right garment”) including similar components to form a pair of temperature-sensing garments (e.g., a pair of left and right garments).

In some variations, the garment can assist in diagnosing and/or predicting conditions in a user. For example, users with diabetic neuropathy (which may cause loss of feeling in users' feet) may be unable to identify a sore or infection formed on their feet and/or periods of poor blood circulation through their feet, which may lead to debilitating infections and, ultimately, to amputation. When a region of a foot becomes infected, the temperature of this region of the foot may rise as the body combats this infection. While a diabetic user may be less able to feel pain from this infection, temperature-sensing garments (e.g., garments, slippers, shoes, shoe inserts, or other soft goods worn by the user) may read temperatures of various regions across the user's feet. The temperature measurements may be used to predict and/or detect this infection, such as before the infection becomes so severe that the user risks losing the infected foot. Exemplary methods for assessing foot inflammation based on foot temperature measurements from the system are described in U.S. Patent App. Pub. No. 2017/0188841, which is hereby incorporated in its entirety by this reference.

In one exemplary variation, the garment 810 may include one or more temperature sensors 820 (e.g., thermistor, thermocouple) arranged in a configuration (or “pattern”) enabling collection of temperature values at multiple target regions of a foot placed within the temperature-sensing garment. For example, a left temperature-sensing garment (hereinafter “left garment”) can include six temperature sensors arranged in a left pattern with: a first temperature sensor 820a positioned in a first region of the temperature-sensing garment configured to face the first Ossa digit proximal the distal phalange of a user's left foot when the left garment is worn by the user; second, third, and fourth temperature sensors (820b, 820c, 820d) positioned across a second region of the temperature-sensing garment configured to face the boundary of the phalanges and the metatarsals of the user's left foot when the left garment is worn by the user; a fifth temperature sensor 820e positioned in a third region of the temperature-sensing garment configured to face the boundary of the metatarsals and the tarsals of the user's left foot when the left garment is worn; and a sixth temperature sensor 820f positioned in a fourth region of the temperature-sensing garment configured to face the heel of the user's left foot when the left garment is worn by the user. The left garment can thus include a set of temperature sensors distributed across four distinct temperature zones of the user's foot. A right temperature-sensing garment (hereinafter “right garment”) can include a set of six temperature sensors arranged in a right pattern mirroring the left pattern.

In other variations, temperature sensors may additionally or alternatively be arranged on other regions of the garment (e.g., medial or lateral sides of a foot region of the garment, dorsal portion of a foot region of the garment, ankle region of the ankle, etc.). For example, one or more temperature sensors may be arranged on the garment so as to measure skin temperature of the ankle and/or leg (e.g., lower leg).

In some variations, the garment may further include at least one housing 840. As shown in FIG. 8B, the housing 840 may house an electronic subsystem for receiving data (e.g., from sensor leads) from the temperature sensors, and/or house other various components. The housing 840 may, for example, be arranged on an ankle region of the garment. In some variations, the housing may be secured to the garment by being enclosed between the garment and a cover. As another example, the garment can include a pocket configured to receive the housing, and channels configured to receive wires extending from the housing to each temperature sensor in the set of temperature sensors. For example, the pocket or pouch can be sewn into a neck or ankle portion (e.g., upper portion) of the temperature-sensing garment during manufacturing. In another example, the garment can include channels sewn along a side of a user's foot when the garment is worn by the user, such that the user may not feel the wires within the garment when the user wears the garment. Additionally or alternatively, the housing may be coupled to the garment with an adhesive or mechanical fasteners (e.g., rivets, snaps, etc.), sewn to the garment, or in any suitable manner.

The housing may be substantially sealed (e.g., hermetically sealed or waterproofed) to protect the contents of the housing from environmental conditions (e.g., when the garment is washed or worn, when the user is sweating, etc.). For example, at least a portion of the housing may be filled with and/or otherwise sealed with a substance adhering to the housing, such as ultraviolet glue, silicone, other epoxy or polymer, etc. As another example, the housing may include one or more components that may be coupled together (e.g., with a suitable mechanical interfit, fasteners, weld, etc.). The joint between coupled housing components may be sealed by epoxy or other suitable sealant. In some variations, the one or more components of the housing may be formed at least in part through injection molding.

As shown in the schematic of FIG. 8B, the housing 840 may house various components for operating the system. For example, the housing 840 may include at least one processor 852 (e.g., CPU), at least one memory device 854 (which can include one or more computer-readable storage mediums), at least one communication module 856, and at least one power source 858. In some variations, the housing may further include one or more additional activity or other sensors (e.g., an accelerometer, a gyroscope, or an inertial measurement unit). One or more of these components may be arranged on one or more electronic circuit boards (e.g., PCBA), which in turn may be mounted to the housing.

The processor 852 and memory device 854 may cooperate to provide a controller for operating the system. For example, the processor 852 may receive sensor data from one or more sensors 824 (e.g., first temperature sensor 824a, second temperature sensor 824b, third temperature sensor 824c, etc.), and the sensor data may be stored in one or more memory devices 854. In some variations, the processor 852 and memory 854 may be implemented on a single chip, while in other variations they can be implemented on separate chips. In some variations, the controller may be similar to the controller described in U.S. Patent Pub. No. 2017/0188841, which was incorporated by reference above. Other exemplary aspects of the controller are described herein.

Generally, the controller can operate in an inactive state and in an active state. The controller may operate in the inactive state when there is an indication that the garment is not being worn by the user, and may operate in the active state when there is an indication that the garment is being worn by the user. In the inactive state, the controller may be in a “sleep” mode (e.g., to conserve energy in the power source 858). The inactive state may, for example, be useful to limit depletion of the power source 585 when the garment is being manufactured, stored (e.g., in a warehouse), and/or shipped or transported. Accordingly, the inactive state can advantageously extend shelf life of the garment. In the active state, the controller may be in an “awake” mode in which sensor data is received, processed, and/or stored in the memory device 154 for use in monitoring for inflammation. For example, in its active state, the controller may scan at least some of the temperature sensors to receive and store temperature measurement data (e.g., periodically, such as every second, every 10 seconds, every 30 seconds, every minute, every hour, or other suitable interval). Furthermore, in some variations, transitioning to the active state (e.g., the first instance of transitioning to the active state) may include registering the garment to a computing device (e.g., hub, smart phone, etc.).

The controller may toggle between the inactive state and the active state based on based on user input (e.g., pressing of a button), sensor data (e.g., from activity sensors, processing of temperature data, etc.) suggesting whether the garment is placed on a user, and/or passage of a predetermined period of time. For example, as further described below, a controller integrated into a temperature-sensing garment can remain in an inactive mode as a default (e.g., based on an assumption that the majority of the time, the temperature-sensing garment is not being worn). However, the controller can “wake” and transition to the active mode based on one or more signal inputs suggesting that the garment is being worn by a user. Variations of methods activation and registration of the garment are described in further detail below.

The communication module 856 may be configured to communicate sensor data and/or other information to an external computing device 870. The external computing device 170 may be, for example, a mobile computing device (e.g., mobile telephone, tablet, smart watch), laptop, desktop, hub such as that described herein, or other suitable computing device. The external computing device 170 may be executing a native application for presenting sensor data (and/or the results of analysis thereof) through a user interface to a user. Additionally or alternatively, the communication module 156 may be configured to communicate to one or more networked devices, such as a hub paired with the system, a server, a cloud network, etc.

The communication module 156 may communicated via a wired connection (e.g., including a physical connection such as a cable with a suitable connection interface such as USB, mini-USB, etc.) or a wireless network (e.g., through NFC, Bluetooth, WiFi, RFID, or any type of digital network that is not connected by cables). For example, devices may directly communicate with each other in pairwise connection (1:1 relationship), or in a hub-spoke or broadcasting connection (“one to many” or 1:m relationship). As another example, the devices may communicate with each other through mesh networking connections (e.g., “many to many”, or m:m relationships), such as through Bluetooth mesh networking. Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSDPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), or any other suitable communication protocol. Some wireless network deployments may combine networks from multiple cellular networks or use a mix of cellular, Wi-Fi, and satellite communication.

Additionally, the housing may include one or more power sources 858, which may function to provide electrical power to the processor, communication module, sensors, and/or any other electrical components. For example, the power source 858 may include one or more batteries. In some variations, the power source 858 may be rechargeable such as through wireless charging methods (e.g., inductive charging, RF coupling, etc.) or by harnessing kinetic energy such as that generated through motion (e.g., when the user walks while wearing the garment).

### Registration and Activation

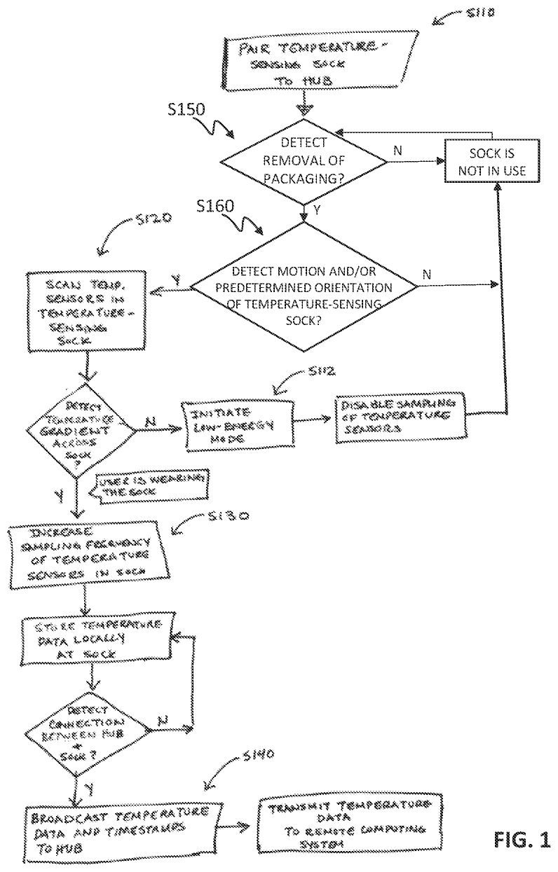

As shown in FIG. 1, a method S100 for registering temperature-sensing garments for detecting medical conditions includes: pairing each temperature-sensing garment in a set of temperature-sensing garments to a hub (S110); and maintaining each temperature-sensing garment in the set of temperature-sensing garments in an inactive mode (S112). The method may further include identifying one or more conditions indicating the garment is being worn by a user. One exemplary condition indicating the garment is being worn by a user is detecting removal of one or more packaging components for the garment (S150) (or separation between the garment and one or more packaging components). Another exemplary condition indicating the garment is being worn by a user is detecting motion of a first garment in the set of temperature-sensing garments (S160). Another exemplary condition indicating the garment is being worn by a user is detecting an orientation of the first garment suggesting its placement on a user (e.g., housing on ankle portion of the garment is upright)) (S160). Yet another exemplary condition indicating the garment is being worn by a user is detecting a first temperature gradient between discrete temperature sensors integrated in the first temperature-sensing garment exceeding a threshold gradient (e.g., when scanning one or more temperature sensors integrated in the first temperature-sensing garment during an initial scan cycle (S120)). Any one or more of such conditions may be relied upon. Furthermore, although FIG. 1 depicts an exemplary order in which these conditions may be relied upon, it should be understood that these conditions may be applied in any suitable order to determine that the garment is placed on a user. After determining the garment is placed on a user, the method may include scanning the discrete temperature sensors continuously at a first sampling frequency over a scan period (S130). The method may also include broadcasting temperature data recorded by the first temperature-sensing garment over the scan period to the hub in response to detecting a wireless connection between the first temperature-sensing garment and the hub (S140).

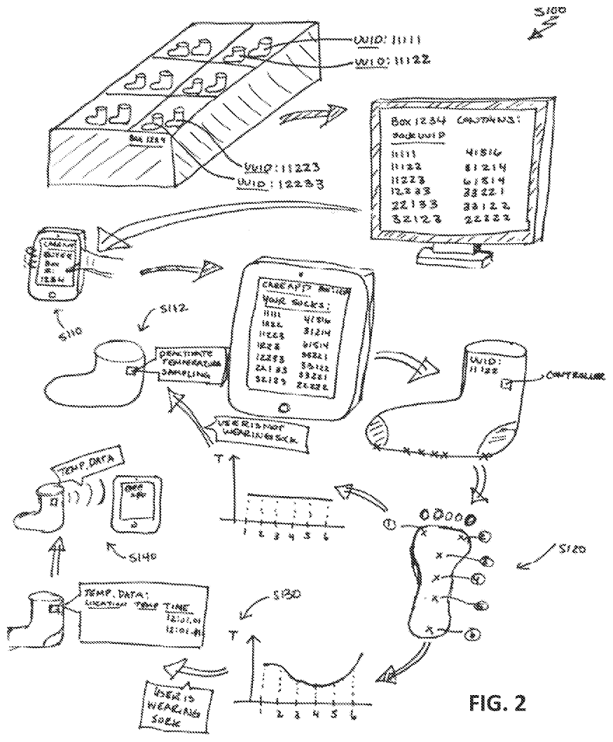

One variation of the method shown in FIG. 2 may further include: linking a unique identifier of each garment in the set of garments to a mobile computing device affiliated with a user; and, in response to detecting a wireless connection between the mobile computing device and the first temperature-sensing garment, broadcasting the temperature data to the mobile computing device.

Generally, the method may be implemented by at least one computing device in cooperation with a temperature-sensing garment containing temperature sensors to: register temperature-sensing garments to a user in possession of the temperature-sensing garments; selectively sample temperature sensors exclusively when the user is wearing the temperature-sensing garment; and efficiently communicate temperature data recorded by the temperature-sensing garment to a remote computer system to detect temperature gradients indicating foot-borne medical or health conditions and notify an appropriate care provider to assist the user. Therefore, the computing device and the temperature-sensing garment may cooperate to facilitate: registration of the temperature-sensing garment to a user, collection of temperature data, and communication of temperature data to care providers to diagnose a foot-borne medical or health condition of the user automatically and in real-time based on the temperature data. Therefore, the computing device and the temperature-sensing garment may cooperate to enable efficient medical response to the user in response to detecting foot-borne medical or health conditions.

In some variations, the temperature-sensing garment may pair to the computing device automatically (or semi-automatically) when the computing device is within range of the temperature-sensing garment. In one implementation, the temperature-sensing garment broadcasts temperature data collected at the temperature-sensing garment to a hub that plugs into a wall outlet. In this implementation, a particular hub may be paired to and/or packaged with the set of temperature-sensing garments at a packaging facility during a final pack-out. Upon receipt of the hub and temperature-sensing garment, a user may plug the particular hub into a wall outlet and unpack the set of temperature-sensing garments. The user may wear a pair of temperature-sensing garments, which can record temperature data of the user's feet while the user wears the temperature-sensing garments. The temperature-sensing garments may store temperature data locally until the pair of temperature-sensing garments return within range (e.g., five meters) of the hub. In response to wireless connection between the hub and the pair of temperature-sensing garments, the temperature-sensing garment may broadcast the temperature data (with corresponding timestamps) to the hub. The hub may transmit temperature data to a remote database over a wireless connection (e.g., an internet and/or cellular connection), which may serve notifications to the user and/or to a care provider in response to detecting temperature changes that indicate a foot-borne condition. Therefore, the temperature-sensing garment may broadcast temperature data to the hub—in lieu of an intervening mobile computing device—to detect temperature changes and, in real-time, notify a care provider or other person of foot-borne medical conditions, such as for users without regular access to a mobile computing device and/or without consistent wireless (internet) connection.

To preserve battery life of the temperature-sensing garment, the temperature-sensing garment may remain in an inactive or “sleep” mode, in which the system deactivates scanning of temperature sensors. However, in response to detecting motion of the temperature-sensing garment and/or detecting a predetermined orientation of the garment, the temperature-sensing garment may sample the temperature sensors integrated into the temperature-sensing garment at discrete locations across the user's foot (e.g., proximal each of the user's toes, proximal a mid-point of the user's arch, and proximal the user's heel) during an initial scan cycle. In response to detecting a temperature gradient across the temperature sensors, the system may initiate an active or “awake” mode, in which the temperature-sensing garment periodically samples the temperature sensors at a predefined frequency (e.g., once every fifteen seconds). However, in response to detecting absence of a temperature gradient across the temperature sensors, the temperature-sensing garment may return to the inactive mode in which the temperature-sensing garment scans the temperature sensors infrequently (e.g., once every thirty minutes).

A user may wear the temperature-sensing garment intermittently (e.g., once every six days) over an extended period of time (e.g., seven weeks). To conserve battery-life of the temperature-sensing garment and extend life span of the temperature-sensing garment, the temperature-sensing garment may selectively disable temperature sensing when the temperature-sensing garment is not being worn by a user. In one implementation, the temperature-sensing garment may scan motion sensors and/or orientation sensors intermittently (e.g., every two minutes) throughout the lifespan of the temperature-sensing garment. In response to detecting motion (i.e., acceleration) of the temperature-sensing garment and/or detecting a predetermined orientation (e.g., upright or vertical) of at least a portion of the temperature-sensing garment, a controller may flag the motion and/or orientation as a potential indicator that the user may be wearing the temperature-sensing garment. In some variations, both conditions of detected motion and detected predetermined orientation may be relied upon to help exclude situations such as shipment (e.g., acceleration but perhaps not vertical orientation of the garment) and storage (e.g., might be vertical orientation but no acceleration) from signaling that the garment is placed on a user.

After detecting motion and/or the predetermined orientation, the method may include scanning temperature sensors intermittently (e.g., once every two minutes) to further confirm whether the user is wearing the temperature-sensing garment. The temperature-sensing garment may detect a temperature gradient between temperature sensors within the temperature-sensing garment, which may be used to identify that the user is wearing the temperature-sensing garment. The temperature-sensing garment may scan the temperature sensors at a higher frequency (e.g., once every ten second) to capture consistent temperature data of the user's foot while the user wears the temperature-sensing garment. Therefore, the controller may selectively—and exclusively—sample temperature sensors when the temperature-sensing garment confirms the user is wearing the temperature-sensing garment.

Therefore, the computing device (e.g., a mobile computing device affiliated with a user and/or a hub configured to pair with the temperature-sensing garment) may: pair to and/or register temperature-sensing garments; link each temperature-sensing garment to a user profile of a user in possession of the temperature-sensing garment; detect when the user is wearing the temperature-sensing garment; and selectively—and intermittently—scan temperature sensors of the temperature-sensing garment when the user is wearing the temperature-sensing garment. Each temperature-sensing garment may, therefore, record temperature changes across the user's foot and store the temperature data locally at the temperature-sensing garment until the temperature-sensing garment establishes a wireless connection with the device. From the temperature data, the computing device may calculate and extract trends from the temperature data to diagnose foot-borne medical conditions.

### Pairing

At least some portions of the method may additionally or alternatively be executed on a computing device in communication with the temperature-sensing garment(s), such as a mobile computing device (e.g., a smartphone, smartwatch, or tablet), a hub (or a beacon or dongle) configured to broadcast data over a wireless and/or cellular network, or any other device communicatively coupled to the temperature sensing garment via wireless communication protocols.

### Pairing: Hub

As shown in FIG. 1, the method may include pairing each temperature-sensing garment in a set of temperature-sensing garments to a hub (S110). Generally, the controller of the temperature-sensing garment may link or pair to a hub (or other wireless communication device configured to transmit and receive data from temperature-sensing garments) such that the controller may intermittently transmit temperature data to the hub, which may transmit relevant temperature data to a remote computer system and selectively alert a care provider to assist the user in response to detecting a temperature gradient corresponding to a medical condition.

In one exemplary implementation, each temperature-sensing garment (and/or each pair of temperature-sensing garments) may be loaded with a unique user ID (e.g., a UUID) and/or mechanical address. Prior to shipment of a set of temperature-sensing garments to a user, an operator may scan and record the UUID for each temperature-sensing garment in a set of temperature-sensing garments (e.g., seven pairs of left and right temperature-sensing garments). The UUIDs of each temperature-sensing garment may be uploaded to the hub, such that the hub receives signals from each temperature-sensing garment and, therefore, pair each temperature-sensing garment in the set of garments to the hub. Accordingly, the hub may transmit data to and receive data from each temperature-sensing garment in the set of temperature-sensing garments within range of the hub.

In the foregoing implementation, the hub may be plugged into a wall and/or stationed within a user's residence and connected to a wireless and/or cellular network. When a temperature-sensing garment is within range (e.g., ten meters) of the hub, the hub may receive a signal from the temperature-sensing garment and route this signal flag, alarm, or notification to a remote computer system and/or network.

In one variation, if the temperature-sensing garment is out of range of the hub (or a second hub configured to receive signals from the temperature-sensing garment), other active temperature-sensing garments (e.g., a left temperature-sensing garment) may cooperate to form a distributed network within the user's residence and can collect and route the flag, alarm, or notification to a nearby wireless communication hub for distribution to the remote computer system.

Additionally or alternatively, the hub may be linked to a user profile hosted by a remote computer system and accessible by the user and/or a care provider (e.g., via a user portal, native application, and/or web browser). In this implementation, by pairing a temperature-sensing garment to a particular hub assigned and/or affiliated with the particular user, the hub may directly import temperature data recorded by each temperature-sensing garment linked to the hub into the user profile.

However, in other variations, one or more temperature-sensing garments may additionally (e.g., as confirmation) or alternatively be paired to a hub after it is determined that the garments are placed on a user.

### Pairing: Computing Device

In one variation, a temperature-sensing garment may pair to a mobile computing device, such as a smartphone, tablet, smartwatch, etc., over a wireless communication network. For example, the mobile computing device may render a native application configured to read the UUIDs of each temperature-sensing garment to pair each temperature-sensing garment to the mobile computing device.

In one implementation, prior to shipment of a set of temperature-sensing garments to a user, an operator may scan and record the UUID of each temperature-sensing garment in a set of temperature-sensing garments (e.g., six pairs of left and right temperature-sensing garments). In this variation, the operator may assign a unique code or identifier (e.g., a QR code) to the set of temperature-sensing garments. For example, a box in which the set of garments are shipped may include a barcode, RFID tag, QR code, and/or any other code (e.g., alphanumeric) to identify the set of garments contained within the box. The UUIDs of each temperature-sensing garment in the set of garments may be paired to the unique code assigned to the set of garments and uploaded to a database (e.g., a remote naming server). Upon receipt of the set of temperature-sensing garments, a user may download a native foot health application (hereinafter the “native application”) to the user's mobile computing device. The native application may prompt the user to: log into a user profile rendered within the native application; scan the unique code of the set of garments (e.g., a QR code on the box in which the set of temperature-sensing garments arrived); and link the unique code of the set of garments to the user's profile. The native application can serve the unique code of the set of garments to the database in order to access (or download) UUIDs for each temperature-sensing garment and link each UUID to the user's profile to pair each temperature-sensing garment to the native application. Once paired with the native application, each temperature-sensing garment may wirelessly broadcast temperature data directly to the native application executing on the mobile computing device (e.g., rather than the hub) when the temperature-sensing garment is within range of the mobile computing device. The native application may then automatically or semi-automatically (e.g., based on user input) upload the temperature data to the user's profile.

Furthermore, a user may wear a left-labeled temperature-sensing garment (hereinafter “left garment”) and a right-labeled temperature-sensing garment (hereinafter “right garment”). A left controller in the left garment and a right controller in the right garment may: sample temperature sensors contained in the left and right garments, respectively, over time; pair wirelessly with a mobile computing device carried by the user as described below; and upload temperature data to the mobile computing device for processing. A native foot health application executing on the mobile computing device may compare and analyze these foot temperature data in real-time and/or over time to identify possible medical conditions present in one or both of the user's feet. In this example, the native foot health application (hereinafter, “native app”) may: calculate a difference between temperatures measured at like or corresponding regions on the user's left and right feet at similar times (e.g., substantially simultaneously); identify a possible medical condition in one of the user's feet if the temperature in that foot exceeds the corresponding temperature in the other foot by a threshold difference; and present a notification to respond to the possible medical condition or to seek medical attention to the user directly through the mobile computing device as described below.

However, in other variations, one or more temperature-sensing garments may additionally (e.g., as confirmation) or alternatively be paired to a computing device after it is determined that the garments are placed on a user. Furthermore, the temperature-sensing garment may pair to the mobile computing device (and/or any other device) in any other suitable way.

### Pairing: Computing Device to Hub

As an extension of the foregoing variation, the mobile computing device affiliated with the user may also pair to the hub, such as via a cellular (e.g., 3G, 5G, LTE, etc.) network and/or a local wireless computer network. In this variation, the native app executed by the mobile computing device may access an address (e.g., mechanical and/or wireless address) of the hub and link the hub to the user profile hosted by a remote computer system. When the temperature-sensing garment broadcasts temperature data (and/or temporal data) to the hub, the hub may automatically (or semi-automatically) upload the temperature data to the user profile. Therefore, the mobile computing device and the hub may cooperate to receive data from the temperature-sensing garment(s) and upload this data to the user profile, which may be accessed by a care provider and/or the user. Furthermore, by linking the mobile computing device and the hub, the native app may link the hub directly to the user profile, such that the hub may directly upload temperature data to the user profile in the absence of the mobile computing device.

For example, a user may access her mobile computing device (e.g., a smartphone) infrequently. Therefore, a temperature-sensing garment worn by the user may selectively broadcast temperature data to a hub paired to the temperature-sensing garment when the temperature-sensing garment is within range (e.g., five meters) of the hub and the hub may upload the temperature data to the user profile. However, if the mobile computing device is active and within range of the temperature-sensing garment, the temperature-sensing garment may broadcast temperature data directly to the mobile computing device.

### Detection of Packaging Removal

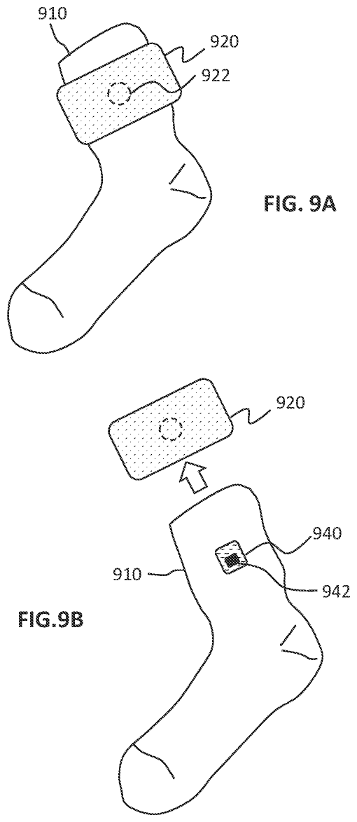

In some variations, as shown in FIGS. 9A and 9B, the method may include detecting removal of packaging (S150) (e.g., separating the garment from packaging). Since packaging removal is a familiar step among consumers, detecting the removal of packaging can be one factor (alone or in combination with other triggers as described herein) used to indicate that a garment associated with the packaging is being prepared for use (e.g., for wearing by a user), without requiring additional or non-intuitive steps by the user. As described herein, the garment may be transitioned from an inactive state to an active state at least partially based on the detection of removal of the packaging. In other words, activation of the garment may, at least in part, leverage intuitive consumer behavior including removal of packaging.

Packaging removal can be detected at least in part by a packaging sensor coupled to the garment. For example, as shown in FIG. 9A, a garment 910 can be associated with packaging 920. The packaging may including labeling and/or protective components for shipping and transport. For example, although the packaging is depicted as a sleeve (e.g., slip on, wrap-around with coupleable ends, etc.) for wrapping at least partially around the garment 910 (or a pair of garments 910), the packaging can additionally or alternatively include a bag, shrink wrap, tissue packaging, tape, padding (e.g., packing peanuts, air bags, etc.), and/or other suitable packaging. The packaging can be made of paper, plastic, or other suitable material. As shown in FIG. 9B, the garment 910 may further include at least one packaging sensor 942 configured to detect removal of the packaging 920. The packaging sensor 942 may be arranged, for example, in the housing 940 (e.g., similar to the housing variations described above).

In one exemplary variation, the packaging sensor 942 may include at least one sensor configured to output a signal corresponding to the presence (or absence) of a magnetic field. For example, the packaging sensor may include a Hall effect sensor, and the packaging 920 may include at least one magnet 922 configured to generate a magnetic field proximate the Hall effect sensor when the packaging is on or near the garment 910. For example, the magnet 922 may be attached to the packaging with adhesive or other suitable fastener, inserted into a pocket in the packaging, laminated between adjacent layers of material in the packaging, etc. The magnet 922 may be a permanent magnet. The packaging sensor 942 may emit a first signal value (e.g., voltage) when the packaging is on or near the garment 910 thereby generating a magnetic field detectable by the packaging sensor 942, and the first signal value may indicate that the packaging has not been removed. Furthermore, the packaging sensor 942 may emit a second signal value when the packaging 920 is separated from the garment 910 thereby removing the magnetic field, and the second signal value may indicate that the packaging has been removed. Accordingly, the packaging sensor 942 may be configured to detect separation of the garment from the packaging based on the interaction between the packaging sensor and the packaging.

In another exemplary variation, the packaging sensor 942 may include at least one light sensor. The packaging sensor may include a photodetector or other suitable light sensor, and the packaging 920 may initially cover the packaging sensor and limit light exposure of the packaging sensor. For example, the packaging sensor 942 may be a white (visible) light sensor and the packaging 920 may be opaque and substantially prevent white light from being incident upon the packaging sensor 942. As another example, the packaging sensor 942 may be configured to detect a selected wavelength or range of wavelengths (e.g., red light) and the packaging 920 may include a filter to substantially prevent light of the selected wavelengths or range of wavelengths from being incident upon the packaging sensor 942. The packaging sensor 942 may emit a first signal value (e.g., voltage) when the packaging at least substantially covers the packaging sensor on the garment, and the first signal value may indicate that the packaging has not been removed. The packaging sensor 942 may emit a second signal value when the packaging 920 is separated from the garment thereby exposing the packaging sensor 942 to light, and the second signal value may indicate that the packaging as been removed. Accordingly, similar to the variation described above, the packaging sensor 942 may be configured to detect separation of the garment from the packaging based on the interaction between the packaging sensor and the packaging.

### Triggers: Motion and Orientation Data

As shown in FIG. 1, the method may include maintaining each temperature-sensing garment—in the set of temperature-sensing garments—in an inactive mode (S112). Generally, the controller integrated into the temperature-sensing garment may remain in an inactive mode in order to limit battery-usage when the temperature-sensing garment is not being worn. However, the controller may wake when one or more sensors in the garment outputs a signal indicating that the garment is in motion and/or a signal indicating that at least a portion (e.g., ankle portion) of the garment is in a predetermined orientation (e.g., upright or vertical), and then sample temperature sensors in the garment. Then, if signals from the temperature sensors indicate that the garment is increasing in temperature, the controller may enter an active state, as described below; otherwise, the controller may return to the inactive state for at least a minimum duration of time (e.g., one minute).

As described above, the temperature-sensing garment may include orientation sensors (e.g., an IMU, an accelerometer, a gyroscope, and/or a compass) configured to detect motion and changes in orientation (S160), which may indicate that the temperature-sensing garment is currently being worn by the user. In one implementation, the orientation sensor includes an inertial measurement unit (or “IMU”) integrated into an anklet or housing of the temperature-sensing garment and configured to measure a change in acceleration. The orientation sensor may couple to an interrupt pin on the controller of the temperature-sensing garment, which may default to a sleep or “low-energy” state in which the controller executes a minimum of processes. Additionally or alternatively, the orientation sensor may detect the current orientation of the garment for comparison to a predetermined orientation corresponding to likely placement of the garment on a user. For example, in variations in which the orientation sensor is located on an ankle portion of the garment, the controller may determine whether the current orientation of the garment substantially matches an upright or vertical orientation (which indicates the ankle portion is placed on a leg of the user) (e.g., equal to the upright orientation within a predetermined threshold). In response to detecting motion above a predetermined threshold and/or detecting a predetermined orientation of the temperature-sensing garment, the controller of the temperature-sensing garment may trigger the controller to transition from the sleep state to an active state. Once in the active state, the controller may sample the orientation sensors (e.g., periodically or intermittently).

Additionally or alternatively, the temperature-sensing garment may include a proximity sensor configured to detect the presence of skin or a body part inside the garment, which may indicate that the garment is currently being worn on a user's foot. In one implementation, the proximity sensor includes a capacitive touch sensor facing the internal surface of the garment and integrated into an anklet embedded into or installed over the exterior of the garment, such as near the mouth of the garment. In this implementation, the anklet may further house a controller, a battery, and a computer-readable memory, similar to that described above. The proximity sensor may be coupled to an interrupt pin on the controller, which can default to a sleep state in which the controller executes a minimum of processes. Proximity to a massive object (e.g., a foot or a leg) can trigger a change in the output state of the proximity sensor from a binary LO voltage to a binary HI voltage (or vice versa), which can trigger the controller to transition from the sleep state to an active state. Once in the active state, the controller can regularly sample the proximity sensor, such as just before each temperature scan cycle or once per ten-minute interval, as described below.

Additionally or alternatively, the controller may switch into the active state in response to receipt of an activation input from the user's mobile computing device (i.e., from the native app). Similarly, the controller may return to the sleep state in response to a change in the output of the proximity sensor to the binary LO voltage, thus indicating that a foot has been removed from the temperature-sensing garment, or in response to a deactivation command received from the user's mobile computing device.

However, the temperature-sensing garment may initiate the active state in response to any other trigger.

### Temperature Data

As shown in FIG. 1, the method S100 may include scanning temperature sensors integrated in the first temperature-sensing garment during an initial scan cycle in response to detecting motion of a first garment in the set of temperature-sensing garments (S120); and, in response to detecting a first temperature gradient between discrete temperature sensors integrated in the first temperature-sensing garment exceeding a threshold gradient, scanning the discrete temperature sensors continuously at a first sampling frequency over a scan period (S130). Generally, a controller integrated into a temperature-sensing garment worn by the user may detect the presence of the user's skin to confirm that the garment is currently being worn, and may scan temperature sensors integrated into the temperature-sensing garment while the controller in the temperature-sensing garment is active.

While in the inactive state, the controller may intermittently scan the temperature sensors (e.g., every two minutes). In one implementation, the temperature sensors output temperatures in the form of analog resistance values that the controller converts into digital temperature values (hereinafter “temperature data”). Additionally, while in the inactive state, the controller may store temperature data in local memory and/or transmit temperature data to the user's mobile computing device via a wireless communication module, such as in real-time or asynchronously when the wireless communication module intermittently connects to the hub and/or the mobile computing device.

The controller may collect temperature data of a foot in the temperature-sensing garment by scanning the set of temperature sensors embedded within the temperature-sensing garment. The controller may scan any number of temperature sensors within the set of temperature sensors at substantially the same time. For example, the controller may scan all of the temperature sensors integrated into the temperature-sensing garment at the same time. In another example, the controller may scan only one of the temperature sensors in the temperature-sensing garment at one time.

A controller may scan temperature sensors from multiple garments. For example, in one implementation, a left controller in a left garment may scan temperature sensors integrated into a right garment (e.g., the second of a pair of garments) with temperature sensors arranged in a pattern mirroring that of the temperature sensors integrated into the left garment. In this implementation, the left controller may scan each of the temperature sensors integrated into both the left garment and the right garment at substantially the same time. In another implementation, the left controller may scan a particular pair of corresponding temperature sensors integrated into the temperature-sensing garments (e.g., a temperature sensor included in the left garment and a temperature sensor included in the right garment that occupy mirrored or corresponding positions within their respective patterns) at substantially the same time. By collecting temperature data from the left garment and the right garment at substantially the same times, the native app may discern, for example, between (i) changes in global temperatures of the user's feet due to motion or changing ambient conditions (or “noise”); and (ii) changes in local temperatures of regions of a foot which may indicate a medical condition of increasing risk to the user.

### Trigger: Temperature Data

As shown in FIG. 1, the method S100 may include scanning temperature sensors integrated in the first temperature-sensing garment during an initial scan cycle in response to detecting motion of a first garment in the set of temperature-sensing garments (S120); and, in response to detecting a first temperature gradient between discrete temperature sensors integrated in the first temperature-sensing garment exceeding a threshold gradient, scanning the discrete temperature sensors continuously at a first sampling frequency over a scan period (S130). Generally, a controller integrated into a temperature-sensing garment worn by the user may detect the presence of the user's skin to confirm that the garment is currently being worn and scans temperature sensors integrated into the temperature-sensing garment while the controller in the temperature-sensing garment is active.

In one implementation, before initiating an initial scan cycle, the controller in the temperature-sensing garment may sample the orientation sensors (as described above) to determine if the temperature-sensing garment is currently being worn by the user. In response to confirmation that the temperature-sensing garment is moving, the controller can initiate an initial scan cycle of the set of temperature sensors. The controller may then scan the set of temperature sensors included in the temperature-sensing garment at a sampling frequency (e.g., once every three-minutes). In response to detecting a temperature gradient between temperature sensors (e.g., one degree difference between a temperature-sensor proximal a heel of the foot and a temperature sensor proximal a toe of the foot), the controller may confirm a user is wearing the temperature-sensing garment. The controller may initiate an active mode in which the controller scans the temperature sensors continuously (or intermittently) at a greater sampling frequency (e.g., once every ten seconds).

However, in response to determination that the temperature-sensing garment is not currently being worn by the user, the controller may return to the inactive state.

In the active mode, the controller may scan the set of temperature sensors included in the garment once per two-minute interval. Alternatively, the controller may scan the set of temperature sensors at variable time intervals. For example, the controller may scan the set of temperature sensors once per hour while the controller determines that the user is at rest, such as indicated by outputs of an accelerometer or other motion sensor integrated into the garment or integrated into the mobile computing device. In this example, in response to determination by the controller (or by the native app) that the user is in motion, the controller may scan the temperature sensors once per five-minute interval. In another example, in response to calculation of a difference between two temperature values—read at substantially similar times from two corresponding temperature sensors in the left and right garments—that exceed a preset threshold value, as described below, the native app may transmit a command to the left and right garments to sample the left and right temperature sensors at an increased rate. The left and right garments may, for example, sample the left and right temperature sensors, respectively, at a rate of once per ten-minute interval (or other suitable interval) by default, but may sample the left and right temperature sensors at an increased rate of once per minute in response to receipt of such a command from the native app and/or in response to initiation of the active mode.

In one variation, the controller may scan the set of temperature sensors in response to an input entered by a user. For example, a user may manually trigger the left and right garments to scan the left and right temperature sensors, such as by selecting a “scan” option within the native app executing on the user's mobile computing device; the native app may then transmit scan commands to the left and right garments. Upon receipt of these scan commands, the left and right controllers may scan the left and right set of temperature sensors, respectively, and then transmit these temperature data back to the mobile computing device for analysis and/or presentation to the user at the native app, such as substantially in real-time.

The controller may also scan the set of temperature sensors to collect a set of control temperature values over a period of time in which the state of the user is known. For example, once a left garment, a right garment, and mobile computing device are wirelessly paired and the native app confirms the battery charge state of the left and right garments, the native app may prompt the user to assume a seated or prone position for a period of time (e.g., five minutes) while baseline or “control” temperature values are recorded. While outputs of an accelerometer or gyroscope integrated in the mobile computing device (or into the left garment or right garment) help confirm that the user is substantially motionless, the left and right garments may regularly sample the left and right temperature sensor, respectively, such as at a rate of 1 Hz, and may upload these temperature data to the mobile computing device. Upon receipt, the native app may label these temperature data as baseline or control temperature values representing a baseline temperature gradient of the user's left foot under constant ambient conditions. Such a temperature gradient may persist across the user's left foot over time even under changing environmental conditions such as walking on garments across a cool tile floor or walking in shoes across a hot asphalt road. Accordingly, as an illustrative example, the native app may compare temperature gradients across temperature values read from the left set of temperature sensors at a later date to the baseline temperature gradient represented by these control data to identify a change in the condition or health of the user's left foot, as described below.

### Broadcasting Data

In some variations, the method may include broadcasting temperature data recorded by the first temperature-sensing garment over the scan period to the hub in response to detecting a wireless connection between the first temperature-sensing garment and the hub. Generally, while in the active state, the controller may store temperature data in local memory. For example, the controller may store up to seven weeks of temperature data in local memory. The controller may transmit temperature data to the user's mobile computing device via a wireless communication module, such as in real-time or asynchronously when the wireless communication module intermittently connects to the user's mobile computing device.

The controller in the temperature-sensing garment may record temperature values read from the set of temperature sensors during a scan cycle and store the temperature data in local memory integrated into the temperature-sensing garment. The controller may also intermittently: pair with the hub; and transmit temperature values stored in local memory to the hub. For example, the controller may transmit temperature values read from the set of temperature sensors to the hub after every fifth scan cycle or once per hour. Alternatively, the controller may transmit temperature values read from the set of temperature sensors to the hub after each scan cycle (e.g., “in real-time”).

In the foregoing implementations, the temperature-sensing garment may communicate with the hub (or other computing device, such as a laptop computer) via a wireless communication channel or over a wireless network. In particular, the controller may transmit data (e.g., temperature, proximity, and/or motion data) sampled from sensors integrated into the temperature-sensing garment to the mobile computing device and may receive data (e.g., commands, scan cycle prompts, clock times, etc. generated by the native app) from the mobile computing device. Furthermore, the temperature-sensing garment (e.g., a “left” garment) may wirelessly pair with a second (e.g., “right”) garment, such as in an instance in which the mobile computing device is not available (e.g., is not within wireless range of the left and right garments). For example, the left garment (or right garment) may maintain an internal master timer and synchronize scan cycles with the right garment or trigger simultaneous scan cycles at the left and right garments, as described above.

### Timestamp

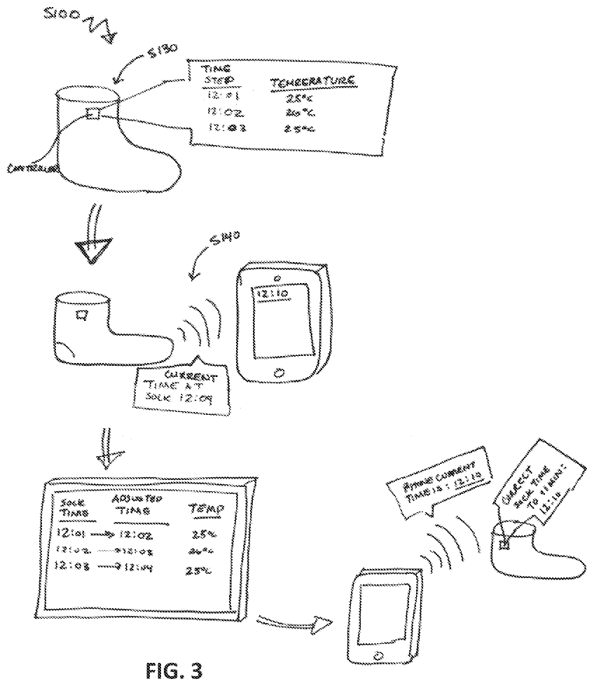

In one variation shown in FIG. 3, the controller may maintain an internal timer that can operate substantially continuously throughout operation. The controller may record timestamps of each temperature value sampled by temperature sensors integrated into the temperature-sensing garment. The controller may broadcast these timestamps to a remote computer system (e.g., a remote server) and/or a mobile computing device.

In one implementation, the controller may pair with the hub and/or mobile computing device, which may access a global (e.g., “atomic”) time over a wireless and/or a cellular network. In this implementation, the controller may calibrate the internal timer to align with the atomic time. However, the internal timer of the controller may drift (e.g., slow or quicken) over time and therefore, a current time recorded at the internal timer may differ from a current atomic time. Therefore, when the controller pairs with the hub and/or the mobile computing device, the controller may query the hub for a current atomic time. The controller may calculate an offset between a current time indicated by the controller's internal timer. For example, the controller may: reset the internal timer to align with the current global time; interpolate offsets for intermediate timestamps; retroactively correct timestamps of temperature values recorded at the temperature sensors; and broadcast the temperature values and corrected timestamps to the hub.

In another implementation, temperature-sensing garments may wirelessly pair with the user's mobile computing device executing the native app, and the native app may synchronize a master clock maintained at the mobile computing device with discrete slave clocks integrated into the left and right garments; once the master and slave clocks are synchronized in time, the left and right garments may intermittently sample the left and right sets of temperature sensors, respectively, such that pairs of temperature data sets generated by the left and right garments throughout their use are substantially matched in time. The native app may compare like-time temperature data sets to reject noise and to identify a medical condition in the user's feet, as described below. (Similarly, the native app may transmit prompts to the left and right garments to scan their temperature sensors for each scan cycle set by the native app.)

In one variation, the hub (and/or native app) may cross-reference UUIDs of temperature-sensing garments broadcasting data to the hub with a registry of UUIDs of temperature-sensing garments paired to the hub and recorded in the user profile as described. In this variation, the hub may reject or ignore temperature data received from temperature-sensing garments with UUIDs absent from the registry as these temperature-sensing garments may be affiliated with another user, such as a spouse or another resident within the same household as the user.

### Duplicate Garment Lockout

In some variations, when a controller in a first left garment transitions into the active state following detection of placement of the first left garment onto a foot, the controller may immediately ping the hub. Upon connecting to the hub, the controller may upload any local data not previously transmitted to the hub and an indication that the first left garment is currently in use. The controller may subsequently regularly broadcast temperature data for the user's left foot to the hub, such as one one-minute intervals. When the controller later determines that the first left garment is no longer being worn (e.g., when signals output by sensors in the first left garment indicate that the garment is no longer moving and is cooling to below typical human body temperatures) and just before the controller returns to the inactive state, the controller may return a prompt to the hub indicating that the first left garment is no longer in use. In this implementation, the hub (or the remote computer system) may also lock out data collected by other garments of the same type (i.e., other left garments)—in the set assigned to the user—from being stored in the user's profile while the first left garment is active. However, once the first left garment returns to the inactive state, the hub (or the remote computer system) may enable collection and storage of temperature data received from a left garment—in the set associated with the user—next activated.

### Pairing Garments

In one variation, pairs of temperature-sensing garments may wirelessly pair with each other. For example, a left garment may wirelessly pair with a right garment, such as in an instance in which the user's mobile computing device is not within wireless range of the left and right garments. In this implementation, a left controller may function as a master controller, and a right controller may function as a slave controller (or vice versa). The left controller may then transmit a scan command to the right garment to trigger each scan cycle such that pairs of temperature data sets are generated by the left and right garments throughout their use and are substantially matched in time. (Similarly, the left garment may synchronize its internal clock with the internal clock in the right garment.)

### Expiration of Garment

In another variation, the temperature-sensing garment may transmit the status of its battery to the hub or native app, which may track the charge state of the battery in the temperature-sensing garment and prompt the user to dispose of the temperature-sensing garment, such as through the graphical user interface and/or haptic feedback (e.g., a vibratory alert) described below when its battery level drops below a threshold level. Furthermore, the controller may be configured to automatically disable temperature sensing and/or other functions of the temperature-sensing garment upon expiration of the battery (e.g., after approximately six months).

The hub or native app may implement similar methods and techniques to manage changes in the battery level of the right garment.

Similarly, if a controller in a garment determines that a temperature sensor in the garment is malfunctioning, the garment may upload all local sensor data to the hub for stored in the user's profile and then automatically disable itself from future use. For example, the garment may transmit to the hub confirmation of malfunction and then disable itself from further temperature monitoring; the hub may receive this prompt from the garment and pass this information to the remote computer system; and the remote computer system may deactivate the garment in the user's profile, trigger shipment of a replacement garment to the user, and/or serve a notification to the user's computing device that the garment has been disabled and that a replacement is on its way.

### Replacement Garments

In one variation, the hub may automatically and wirelessly pair with a replacement temperature-sensing garment (or replacement set of temperature-sensing garments) shipped to the user to replace old (and/or expired) temperature-sensing garments, thereby enabling the replacement garment to upload temperature data to the user profile directly upon receipt of the temperature-sensing garment without additional pairing operations.

In one implementation, upon receiving a replacement garment, the user may open the native app. The native app may then scan a unique code (e.g., QR code), such as arranged on a box containing the replacement garment. As described above, the native app may link a UUID of the replacement garment to a user profile. The temperature-sensing garment may subsequently broadcast temperature and temporal data to the native app, which can upload the temperature and temporal data to the user profile. Additional processes such as those described herein may be performed with respect to the replacement garment.

In another implementation, the user may order a replacement garment (or set of replacement temperature-sensing garments) through the native app or through a web portal rendered on another computing device linked to the profile of the user. Prior to shipment of the replacement garment, an operator may scan a unique code (e.g., QR code) arranged on a box to access UUID(s) of replacement garments within the box. The operator may upload the unique code and the UUID of the replacement garment to the user profile (as denoted in the user's order of the replacement garment) and ship the replacement garment to the user. Upon receipt of the replacement garment, the replacement garment is already paired to the user profile and, thus, is paired to the hub. Therefore, the manufacturer may send replacement garments without sending additional replacement hubs paired to the replacement garments prior to shipment.

### Notifications

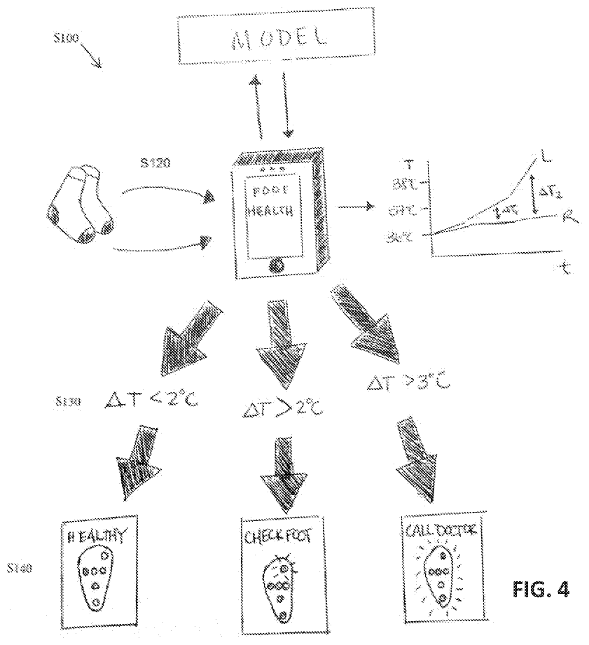

As shown in FIG. 4, a native app—e.g., rendered on a mobile computing device affiliated with the user—may present foot temperature data, foot condition diagnoses, garment function data, and/or related information to the user through a user interface executing within the native app. For example, the native app may display virtual representations of a left foot and/or a right foot overlaid with regions. These regions may be color-coded according to the absolute or relative temperature value read from temperature sensors in corresponding positions within the left and right garments. In this example, the native app: may render green circles over each region of the virtual left foot and right foot corresponding to temperature sensors from which temperature values fall within a first temperature range associated with a healthy condition; and may render red circles over each region of the virtual left foot and right foot corresponding to temperature sensors from which temperature values fall within a second temperature range associated with an unhealthy condition or a medical risk exceeding a preset risk threshold. In some variations, the native app can prompt the user to manually inspect regions of her feet corresponding to red overlay circles on the virtual left foot and right foot shown in the graphical user interface and/or to consult medical attention.

In a similar example, the native app: may serve textual descriptions of visual symptoms of a skin or tissue condition in a submenu corresponding to a region of the virtual left foot and right foot; may prompt the user to select textual descriptions of symptoms that describe the visual state of this region of the user's foot; and/or may respond to such feedback to confirm the predicted medical condition, such as by updating a medical condition model for the user or by escalating a notification to a doctor, nurse, emergency personnel, or other care provider to respond to a confirmed medical condition.

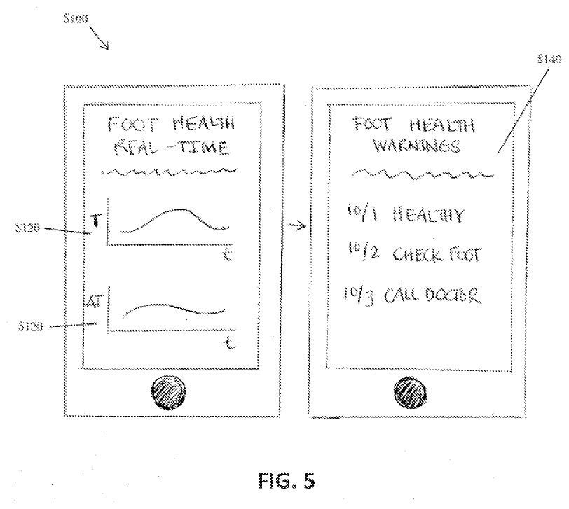

As shown in FIG. 5, the native app may additionally or alternatively render a virtual graph showing absolute or relative (or “normalized”) temperatures of each measured region of the user's feet or an average temperature of each of the user's feet over time. For example, the native app may serve temperature data to the user through a virtual graph in order to visually communicate to the user a trend in temperature of the user's feet over hours, days, weeks, or months, which may indicate a change in the health of the user's feet. For example, the native app may update the graph in real-time as data is received from sensors in the left and right garments.

However, the native app may serve temperature data, medical diagnoses, and/or prompts for user feedback to the user in any other way through the graphical user interface at the user's mobile computing device. Furthermore, the native app may upload temperature data to a remote computer system for remote processing and analysis; the remote computer system may implement similar methods and techniques to process these data and can return medical diagnoses and/or prompts for user feedback to the native app for presentation to the user.

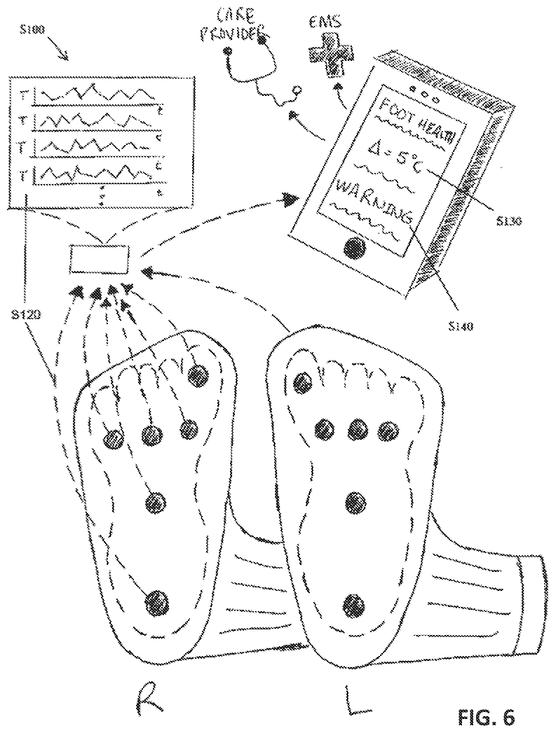

In one variation shown in FIG. 6, the native app may push temperature sensor data and/or medical condition predictions to a remote care provider, such as to a computing device or care provider portal associated with a nurse, doctor, or emergency responder. A care provider may thus view temperature data and/or medical condition predictions for the user's feet in (near) real-time through the care provider portal and respond accordingly, such as by: visiting the user (e.g., for the care provider and user occupying an assisted living facility, hospital, or clinic); calling the user via telephone; scheduling an appointment with the user at a doctor's office; or dispatching an emergency responder to collect the user from the user's current location. Following the care provider's observation of the user, the care provider portal may implement methods and techniques as described above to collect feedback relating to the state of the user's feet from the care provider.

The native app may also write temperature sensor data and/or medical condition predictions directly to an electronic health record associated with the user and stored in a local or remote database.

The systems and methods described herein may be embodied and/or implemented at least in part as a machine configured to receive a computer-readable medium storing computer-readable instructions. The instructions may be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment may be embodied and/or implemented at least in part as a machine configured to receive a computer-readable medium storing computer-readable instructions. The instructions may be executed by computer-executable components integrated by computer-executable components integrated with apparatuses and networks of the type described above. The computer-readable medium may be stored on any suitable computer readable media such as RAMs, ROMs, flash memory, EEPROMs, optical devices (CD or DVD), hard drives, floppy drives, or any suitable device. The computer-executable component may be a processor but any suitable dedicated hardware device may (alternatively or additionally) execute the instructions.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A system for monitoring a user, the system comprising:
a garment configured to be placed on a foot of the user;
a packaging sensor coupled to the garment and configured to detect separation of the garment and packaging associated with the garment; and
at least one processor configured to transition the garment from an inactive state to an active state at least partially based on the detection of separation of the garment and the packaging.

2. The system of claim 1, wherein the garment comprises at least one temperature sensor to measure at least one temperature on the foot of the user.

3. The system of claim 2, wherein the garment comprises a sock and a plurality of temperature sensors to measure temperatures on multiple locations of the foot of the user.

4. The system of claim 2, wherein the garment further comprises a power source, and wherein the at least one processor is configured to reduce use of energy from the power source when the garment is in the inactive state.

5. The system of claim 2, wherein the at least one processor is configured to receive temperature data from the at least one temperature sensor when the garment is in the active state.

6. The system of claim 2, wherein the garment is paired with an electronic device such that the garment communicates temperature data to the electronic device when the garment is in the active state.

7. The system of claim 1, wherein the packaging sensor comprises a Hall effect sensor and the packaging comprises at least one magnet configured to generate a magnetic field proximate the Hall effect sensor.

8. The system of claim 1, wherein the packaging sensor comprises a light sensor configured to detect light when the garment is separated from the packaging.

9. The system of claim 1, wherein the packaging comprises a sleeve configured to wrap at least partially around the garment.

10. A method for operating a temperature-sensing garment, the method comprising:
operating the garment in an inactive state;
detecting separation of the garment and packaging associated with the garment;
predicting that the garment is placed on a user; and
transitioning the garment from the inactive state to an active state in which the garment is configured to measure at least one temperature of the user.

11. The method of claim 10, wherein the inactive state is a low-energy use state compared to the active state.

12. The method of claim 11, wherein the garment is operated in the inactive state during at least one of storage and transport of the garment.

13. The method of claim 10, wherein detecting separation of the garment and packaging comprises detecting removal of a magnetic field.

14. The method of claim 10, wherein detecting separation of the garment and packaging comprises detecting light when the garment is separated from the packaging.

15. The method of claim 10, further comprising pairing the garment with an electronic device.

16. The method of claim 14, further comprising communicating temperature data to the electronic device when the garment is in the active state.

17. The method of claim 10, wherein predicting that the garment is placed on a user comprises detecting at least one of motion and a predetermined orientation of the garment.
